(19) United States
(12) Patent Application Publication
(10) Pub. No.: US 2011/0219686 A1
(43) Pub. Date: Sep. 15, 2011

(54) PLANT GROWTH REGULATOR COMPOSITIONS AND METHODS

(76) Inventor: Volker Heide, Memphis, TN (US)

(21) Appl. No.: 13/046,569

(22) Filed: Mar. 11, 2011

Related U.S. Application Data
(60) Provisional application No. 61/313,428, filed on Mar. 12, 2010.

(51) Int. Cl. A01G 1/00; A01N 43/50; A01N 55/02; A01P 21/00; C07F 5/06; C07F 15/02; C07F 1/08; C07F 15/06; C07F 7/28; C07F 3/02; C07F 11/00; C07F 5/00; C07D 233/70; C07D 401/04; A01D 43/00

(52) U.S. Cl. 47/58.1 R; 504/275; 504/191; 504/253; 504/247; 504/277; 548/101; 548/316.4; 546/2; 546/10; 546/167; 546/274.4; 56/1

(57) ABSTRACT

Described are compositions and methods for regulating plant growth that use a complex of an imidazolinone ion and a metal counterion; the compositions and methods include methods of regulation plant growth, optionally in combination with herbicidal, fungicidal, and fertilizer compositions and effects.

### CROSS-REFERENCE TO RELATED APPLICATION

This application claims the benefit of U.S. Provisional Patent Application Ser. No. 61/313,428, filed Mar. 12, 2010, entitled PLANT GROWTH REGULATOR COMPOSITIONS AND METHODS, the disclosure of which is incorporated herein by reference.

### FIELD OF THE INVENTION

The invention relates to plant growth regulator, herbicide, fungicide, and fertilizer compositions that include imidazolinone ion and an ion such as a metal, organic, or inorganic ion, e.g., as a complex, and methods that use these compositions to control growth of selected plant species, optionally and preferably without the negative effect of widely visible phytotoxicity, while providing residual weed control and enhanced fertilizer and fungicidal effects.

### BACKGROUND

Plant growth regulators are an important class of agricultural chemical. Turfgrasses that provide vegetative cover are widely used on countless commercial and residential applications, to name a few: commercial and residential lawn cover, golf courses, athletic fields, parade grounds, parks, roadsides. Turfgrasses are a cost-effective, conventional, accepted means of controlling erosion and generating esthetic appeal.

Turfgrass is conventionally mowed to maintain a desired appearance of a mowed turf. Grasses are usually mown to reduce leaf height, to remove seedheads, and to produce a uniform, neatly-groomed look. The cost of mowing is expensive in terms of labor and equipment and may add up to several hundred dollars per acre annually. Methods and materials of reducing mowing costs while still maintaining desired aesthetics of turf areas, would have high commercial value due to reduced labor costs, reduced equipment and equipment maintenance costs, and reduced pollution and other emissions, such as carbon emissions. Compounds that act as plant growth regulators, effective to reduce the development of seedheads and inhibit the vertical growth (plant height) of turf swards, may be useful to reduce turf growth, thereby reducing the need for mowing. See, for example, the report “Plant Growth Regulators' Effect on Growth of Mixed Cool-Season Grass Stands at Fort Drum,” by ANTONIO J. PALAZZO, PAUL ZANG, ROBERT W. DUELL, TIMOTHY J. CARY AND SUSAN E. HARDY, September 1996. That report describes evaluations of the effectiveness of two plant growth regulators (PGRs), mefluidide (Embark) and imidazolinone (Event).

Imidazolinones are a class of compounds known to be useful agriculturally active materials, with various utilities in herbicide, pesticide, and plant growth regulator applications. Examples of commercially available imidazolinone products include those sold under the trade designations “ARSENAL”, “CHOPPER”, “STALKER” (imazapyr); “SCEPTER”; “IMAGE” (imazaquin); “ASSERT” (imazamethabenz); “CADRE”, “PLATEAU” (imazapic); “PURSUIT” (imazethapyr); “RAPTOR”, “ODESSEY” (imazamox). Imidazolinone herbicide compounds are understood to be available in a variety of chemically different forms including imidazolinone acid and imidazolinone salts. Examples of herbicidal compositions that can include imidazolinone compounds are described, e.g., in U.S. Pat. No. 6,721,177.

Agricultural chemicals such as herbicides, pesticides, fertilizers, certain growth regulators, etc., are applied to turf to improve the appearance and management of the lawn. A detrimental effect of applying an agricultural chemical to turf (e.g., lawns) is phytotoxicity, or the toxic effect of an agricultural compound on plant growth. Phytotoxicity, as can be caused by imidazolinone and other herbicide materials, can be observed as any one or more of discoloration such as visual browning or yellowing, stunting, or burning of the turf.

### SUMMARY

This invention involves fields of chemical and biological technologies. In specific the invention relates to the use of imidazolinone materials in the form of a complexation with an ion, which may be a metal or a non-metal ion, wherein the imidazolinone can act to selectively inhibit the growth of turf, especially over a period of time. The invention also relates to compositions useful in these methods, and methods of preparing the compositions.

The invention identifies that agricultural chemicals in the class of imidazolinones can be useful when applied to turfgrass species, to result in long term growth regulation (including seed head suppression), and optional pre- or post-emergent weed control. The imidazolinone, in its ionic form, can form a complex with a cation such as a multi-valent metal (e.g., Cu⁺²) or another metal or non-metal counterion. The resultant complex results in a stabilization and deactivation of the imidazolinone ion; the stabilized complex of the imidazolinone ion and counterion is not substantially effective for plant growth regulation, but is substantially stable in a turf environment, i.e., soil or dirt, or the leaf surface itself. Dissociation of the complex in the turf environment releases active imidazolinone ion, which is substantially effective for plant growth regulation and optionally weed control. The process of complexation of the imidazolinone application of the (inactive) complex to turf, and gradual subsequent release of the (active) imidazolinone ion in the turf environment, affects in a positive manner the ability of the active imidazolinone ion to be available in the soil, resist degradation, and become available at different intervals (gradually over a release period) to result in extended plant growth regulation and optional weed control.

As used herein, a “release period” is a period of time after application of the complex to turf and during which the inactive complex dissociates into counterion and (active) imidazolinone ions; the “release period” can correspond to a period of time during which an amount of active imidazolinone ion from the gradually dissociating complex is released into turf and surrounding soil and is present in the soil and turf at a level (e.g., concentration) that is effective to inhibit plant growth. A release period that results in a concentration of active imidazolinone ion that is effective to inhibit growth of turf can be any desired and useful amount of time, for example a period of greater than 2 weeks, such as a period of greater than 3 or 4 weeks, optionally up to or greater than 5, 6, 7, or 8 weeks.

A related potential benefit of a slow or gradual release of the active imidazolinone ion by application in the form of the complex, can be reduced phytotoxicity of the imidazolinone ion against the turf. In preferred embodiments, a cation used to form the complex can function as a safener for the imidazolinone ion against initial turf phytotoxicity as exhibited by any one of: discoloration, stunting, visual browning or burning of the turf. In practical effect, the complex does not exhibit phytotoxicity due to its reduced biologic activity, and the gradual release of the active imidazolinone ion exhibits reduced phytotoxicity relative to a the direct application of a similar amount of imidazolinone ion to turf.

According to the invention, an imidazolinone ion and a cation (or “counterion”) can form a complex in equilibrium with their corresponding ions. A composition that contains the complex can be applied to turf. The imidazolinone ion can be gradually released from the imidazolinone-counterion complex over time, resulting in a residual, gradual, or timed release of the active imidazolinone ion. This gradual release results in gradual and sustained activity of the imidazolinone ion over time and residual biological effect, such as residual regulation of the growth of the turf, optionally also residual weed control effect. Both of these effects occur over a time during which imidazolinone ion is gradually released from the complex, after the complex has been applied to turf and adjacent soil. Preferably, concurrent with the extended release of the imidazolinone can be a safening effect in the form of reduced phytotoxicity to the turf due to the regulated amount of imidazolinone available to the turf at a given time.

In one specific embodiment, a method of the invention can involve the effective use of imidazolinone as a plant growth regulator for turf. The plant growth regulator effect of the imidazolinone is extended over a period of time, during which reduced maintenance of the turf will be required, such as reduced lawn-care in the form of mowing and a reduction in the amount of water needed. As a consequent advantage, a reduced requirement for mowing the turf can result in the use of reduced amounts of energy, e.g., in the form of burning a fossil fuel, providing a relative reduction in a carbon footprint associated with mowing and turf maintenance. Preferred compositions and methods are able to provide extended (e.g., residual) plant growth regulator effect and optional weed control, while avoiding one or more negative effect such as phytotoxicity and root damage as measured by any of: mass reduction, pruning, structural damage, discoloration, or cosmetic damage, by regulating the release the active imidazolinone ion from the complex.

An aspect of the invention is a method of regulating growth of turf by application of a plant growth regulator composition. The method includes: providing a plant growth regulator composition comprising a complex of imidazolinone ion and counterion (which may be a metal or a non-metal ion associated with the imidazolinone ion), and applying the plant growth regulator composition to turf in an amount effective to inhibit growth of the turf.

In another aspect the invention relates to a turf growth regulating composition that includes imidazolinone ion and counterion as a complex.

### DETAILED DESCRIPTION

Methods and materials as described relate to the use of imidazolinone materials in ionic form, and in the form of one or more imidazolinone ion as a complex with a cation such as a metal cation (the “complex”).

Imidazolinone compounds are biologically active in their ionic forms as plant growth regulators for turfgrass, as a herbicide, or as both. An imidazolinone ion can form a complex with a cation (i.e., “counterion” or “stabilizing counterion”) such as a non-metal cation or a metal cation (e.g., Cu⁺²), such that the complex is not biologically active. The equilibrium between the biologically-inactive complex and the active imidazolinone ion and counterion (a metal cation, as illustrated) can be represented by Equation I:

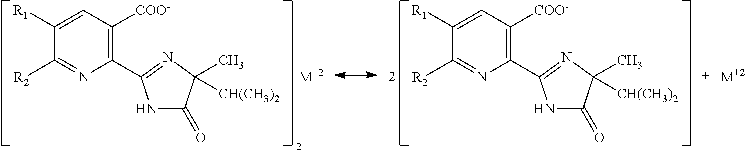

Formation of the complex between the biologically active ionic form of the imidazolinone ion, and a counterion (e.g., a divalent metal cation as illustrated) results in stabilization and deactivation of the imidazolinone ion. The stabilized complex of the imidazolinone ion and counterion (cation) is not substantially effective for plant growth regulation (or other effects such as herbicidal activity) but is substantially stable in a turf environment, i.e., soil or dirt. According to methods as described, the complex is applied to a turf environment and becomes placed in and retained by the turf or adjacent soil. The complex remains in the soil and accessible to the turf over a period of days or preferably weeks. Gradual dissociation of the complex into its ionic components, during this time, within the turf environment, results in gradual release of the biologically active imidazolinone ion. The process of complexation of the imidazolinone ion with a stabilizing counterion, application of the complex to turf, and gradual subsequent release of the imidazolinone ion in the turf environment, allows the active imidazolinone ion to be residually available (i.e., to become gradually available) in the soil for extended growth inhibition. The active imidazolinone ion becomes gradually available over a release period to result in extended (or residual) activity of the imidazolinone ion such as for plant growth regulation and optional weed control. Complexation may also make the imidazolinone ion more resist to degradation.

The rate at which the imidazolinone ion releases from the complex can depend on various factors and can be affected and selected (e.g., controlled) by consideration of those factors. Factors include the chemical identity of the imidazolinone ion; the chemical identity and valency of the stabilizing counterion (e.g., metal ion such as divalent copper); the relative amount of imidazolinone ion compared to the counterion; pH; temperature; environment, such as the type and content of soil; and the presence of other ingredients such as water, surfactant, and other ions. These factors can be assessed and controlled to design an application composition that results in a desired rate of release of imidazolinone ion from the complex after application to soil.

Optionally, to provide predictable or controlled release of imidazolinone ion from the complex, ingredients of an application composition or precursor composition can be selected to prevent interference (by other ionic materials in the composition) with the equilibrium between the complex and the imidazolinone ion and counterion. That equilibrium is important because after application of an application composition that includes the complex, the complex desirably remains present in turf for a period of days or weeks, and gradually releases active imidazolinone ion. For predictable release, the presence of adventitious ions that might interfere with predictable, controlled, gradual, and sustained release of the active imidazolinone ion from the complex may be reduced or minimized. Ions other than the imidazolinone ion and the stabilizing counterion in the complex may be added to a composition in the form of: a counterion to the imidazolinone ion as added to the composition (e.g., the imidazolinone ion may be added to the composition as a salt); a counterion of the counterion of the complex as added to the composition (e.g., the counterion of the complex may be added to the composition as a salt); or may be adventitiously present in another ingredient of a composition such as a solvent, water (e.g., hard water), or as part of another raw material. To allow predictable release of active imidazolinone ion from the complex, a composition can be prepared to avoid, reduce, or minimize the presence of other ions that might have a strong affinity for either the imidazolinone ion or the stabilizing counterion.

Imidazolinone compounds, and chemical derivatives thereof, are a known type of active herbicide compound. Imidazolinone compounds are a class of highly potent, environmentally benign, crop selective herbicides that act as acetolactate synthase inhibitors. Imidazolinone herbicides can be effective for selective control of different varieties of grass and broadleaf weeds in the presence of agricultural crops, and in non-crop areas such as turf forestry. Imidazolinone is generally available in either the acid or salt form. Examples of commercially available imidazolinone salts include alkali metal salts such as sodium salt, a potassium salt; methyl salts; an isopropylammonium salts, among others.

Imidazolinone compounds in their active ionic form can include compounds represented by the following structure:

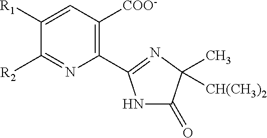

wherein: R1 is hydrogen, methyl, ethyl or a —CH₂—OCH₃ group; R₂ is hydrogen; or R₁ and R2 taken together form a —CH═CH—CH═CH— group.

Some specific examples of imidazolinone materials include salts or ions formed imidazolinone acid materials that include imazethapyr acid, imazaquin acid, imazapyr acid, imazamethabenz acid, imazapic acid, imazamox acid, imazaquin acid, and others. Imazethapyr acid has the molecular formula C₁₅H₁₉N₃O₄ and is commercially available generally in the form of a powdered solid, under the trade designation “PURSUIT.” Imazaquin acid has the molecular formula C₁₇H₁₇N₃O₃ and is commercially available generally in the form of a powdered solid, under the trade designation “SCEPTER” and “IMAGE.” Imazapyr acid has the molecular formula C₁₃H₁₅N₃O₃ and is commercially available generally in the form of a powdered solid, under the trade designation “ARSENAL,” “CHOPPER,” and “STALKER.” Imazamethabenz acid has the molecular formula C₁₅H₁₈N₂O₃ and is commercially available generally in the form of a powdered solid, under the trade designation “ASSERT.” Imazapic acid has the molecular formula C₁₄H₁₇N₃O₃ and is commercially available generally in the form of a powdered solid, under the trade designation “CADRE” and “PLATEAU.” Imazamox acid has the molecular formula C₁₅H₃₉N₃O₄ and is commercially available generally in the form of a powdered solid, under the trade designation “RAPTOR” and “ODESSEY.” While these specific imidazolinone compounds are mentioned, any form of these compounds can be useful as described, including salts, esters, alternate benzene or non-benzene ring transformations. Any chiral form of imidazolinone ion can also be used, such as in a concentrated form (either an S or a R isomer). Accordingly, the composition may include a chiral compound, either S or R isomer, in a predominant amount, e.g., greater than 75 percent or greater than 90 percent.

A counterion of the complex (sometimes referred to as a “stabilizing counterion”) can be any counterion that can become associated with an imidazolinone ion to produce a stable complex that can dissociate in a turf environment to gradually release biologically active imidazolinone ion as described, e.g., to provide residual plant growth regulator activity, and optionally one or more of: weed control, reduced phytotoxicity, reduced root damage, reduced structural damage, and reduced decomposition of the active imidazolinone compound. The stabilizing counterion can be any counterion, especially a cation such as a metal cation that results in stabilization of the active imidazolinone ion as described herein. Certain preferred counterions are divalent, trivalent, or tetravalent cations of metals including Cu, Fe, Mn, Mg, Ca, Ti, Cr, Co, Ga, Al, and any other naturally occurring or man-made di-, tri-, or tetra-valent metal cations, including cations of elements considered to be transition elements. If the stabilizing counterion is a divalent metal cation (represented by M⁺²), a complex can have the following general structure:

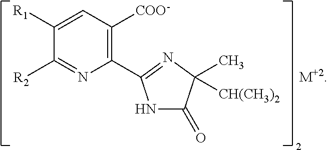

With the metal specifically being divalent copper, the complex can have the general structure:

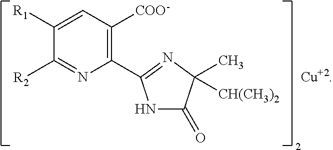

Methods described herein involve application of plant growth regulator compositions to turf, as described, the plant growth regulator compositions including the described complex of imidazolinone ion and stabilizing counterion such as a metal cation, optional amounts of imidazolinone in other forms such as an acid or salt, and optionally an additional amount of the counterion as a free cation or in association with a different counterion (e.g., as part of a metal salt). The term “plant growth regulator composition” refers to a composition that includes a plant growth regulating compound, specifically here, an imidazolinone ion, in combination with a cation that can act as a stabilizing counterion to the imidazolinone ion to form a stable and inactive complex as described. The composition can be in any form, such as an aqueous form (e.g., solution, emulsion, microemulsion, suspension, or the like), a dry non-aqueous form such as a dry salt form (e.g., powder); a solvent-based solution or other non-aqueous liquid; or a mixture of dry and solid forms such as a suspension concentrate. The composition may be useful purely as a plant growth regulator composition, or may be additionally useful as one or more of a herbicide, fungicide, or fertilizer, in which case the composition can optionally include one or more additional biologically active material such as a herbicide, pesticide, fungicide, fertilizer, etc.

Composition as described (“plant growth regulator compositions”) can be of a type designed to be applied directly to turf, or of a type designed to be combined with other ingredients to form a derivative herbicide composition for application (e.g., “application composition”). A plant growth regulator composition having a concentration of the complex that would normally be applied to turf for the purpose of inhibiting growth can be referred to herein as an “application composition,” which refers to a composition for application, as opposed, for example, to a composition having a higher concentration of the complex, which could occur in preparation, storage, shipping, or sale of a herbicide composition.

Amounts of imidazolinone ion, counterion (e.g., divalent metal cation) and the complex, in a plant growth regulator composition, can be any amounts that allow the composition to be applied to turf for effective use as described. Amounts of each included in a plant growth regulator composition in a form applied to turf (i.e., an application composition) can be any convenient amounts, such as amounts that include a suitable concentration of ingredients for application at a desired rate, e.g., based on amount (mass or volume) per acre. The concentration of ingredients in an application concentration might be lower, optionally and typically, that the concentration of ingredients in a precursor, which may be considered any form of a plant growth regulator composition that is not applied directly to turf, but that may be a form for sale, transport, or distribution. These precursor compositions generally include concentrations (e.g., moles or mass of an ingredient, per volume or mass of the precursor) of imidazolinone ion, counterion (e.g., divalent metal cation) and the complex, that are higher concentrations compared to the concentrations of these ingredients in an application composition.

While absolute amounts of imidazolinone ion, counterion (e.g., divalent metal cation) and the complex, in a plant growth regulator composition (e.g. precursor composition, application composition, or other form) can vary based on the purpose of the composition, relative amounts of these ingredients may be within similar ranges in any of the different potential types of plant growth regulator compositions. In this respect, relative amounts of these ingredients can be expressed in parts by weight of each ingredient relative to parts by weight of another ingredient, and the concentrations of each of the separate ingredients (in the composition) may vary widely while the relative amounts of ingredients may remain in a staged relationship.

Functionally stated, relative amounts of imidazolinone ion, counterion (e.g., divalent metal cation) and the complex, in a plant growth regulator composition, can be any amounts that allow the composition to be applied to turf, with the active imidazolinone ion being present after application to function as a plant growth regulator, optionally also as a herbicide. Preferred plant growth regulator compositions can also contain amounts of these components that, when a plant growth regulator composition is applied to turf, can be effective as a plant growth regulator and optionally a herbicide, while avoiding one or more negative effects such as phytotoxicity, root damage, structural damage, mass reduction or pruning, discoloration or other cosmetic damage, etc. Particularly preferred compositions can be applied and can be effective to regulate (inhibit) or substantially prevent growth of turf for a matter of weeks, such as at least 2 weeks, more preferably at least 4, 6, or 8 weeks.

Any relative amounts (molar amounts) of imidazolinone ion and counterion (e.g., metal cation) can be useful in a composition as described. Relative amounts of each ion of the complex in any particular composition can be within a very broad range and can depend on factors such as any one or more of the following: the chemistry of imidazolinone ion and cation (e.g., the particular imidazolinone compound, the particular counterion, and the relative affinity of these ions for each other); the desired release period or application rate; stability of a composition; other components in a composition (e.g., surfactant); the type of composition (e.g., dry, aqueous, liquid); and others. For a divalent metal counterion (such as, but not necessarily, divalent copper) a desired ratio of imidazolinone ion to divalent metal counterion may be up to about 2 imidazolinone ions per divalent metal counterion. Higher relative amounts of counterion (e.g., divalent iron or any other multi-valent metal or non-metal counterion) per imidazolinone ion may also be useful, e.g., up to a molar ratio of 1:5 (imidazolinone ion to counterion), 1:16, 1:32, or up to a molar ratio of 1:64 (imidazolinone ion to counterion). By including imidazolinone ion at a molar ratio such as any of these, a composition can include an amount of imidazolinone ion that is in a free (active) ionic state, and also an amount of imidazolinone ion that is part of the inactive complex with the counterion (e.g., metal cation). Likewise, when used in these ratios, an amount of stabilizing counterion in the composition include an amount in a free ionic state as well as an amount that is part of the complex with the imidazolinone ion.

In different terms, the relative amounts of imidazolinone ion and counterion can be measured based on their relative weights. Examples of relative amounts of imidazolinone ion and counterion (such as a metal counterion) in an application composition or a precursor can be: from 1 to 10 parts by weight imidazolinone ion (such as from 2 to 5 parts by weight imidazolinone ion), per 0.1 to 5 parts by weight counterion (e.g., metal ion) (such as from 1 to 3 parts by counterion (e.g., metal ion)) (each of these can be based on 100 parts by weight application composition).

Amounts of the imidazolinone ion and counterion can also be expressed in terms of a defined amount of the imidazolinone ion and/or counterion in a composition, such as an aqueous composition. For example, the imidazolinone ion and/or counterion can be expressed as the concentration in a liquid composition in terms of weight-volume (e.g., g/L), molarity (e.g, mmol), or parts per million (ppm). In some aspects, the counterion is present in the composition in an amount greater than about 250 mg/L (250 ppm), greater than about 375 mg/L, or greater than about 500 mg/L. (The following molarities of divalent cations at 250 mg/L are noted: 6.25 mmol Ca⁺⁺; 4.48 mmol Fe⁺⁺; 3.93 mmol Cu⁺⁺).

In some aspects, the imidazolinone ion is present in a composition in an amount greater than about 500 mg/L, or greater than about 1 g/L. Molecular weights of imidazolinone ions range from about 260.28 to about 310.3. (For example, the imazapyr ion has a molecular weight of 260.28; the imazapic ion has a molecular weight of 274.3; the imazamethabenz ion has a molecular weight of 287.34; the imazethapyr ion has a molecular weight of 288.3; the imazamox ion has a molecular weight of 304.3; and the imazaquin ion has a molecular weight of 310.3.)

Other ingredients may be used to prepare useful plant growth regulator compositions as described, including readily available adjuvants such as surfactant (e.g., dispersant or other surface active agent), thickener, and antifoaming agent, etc. Additional biologically active ingredient may also be included, such as a fungicide, herbicide, pesticide, or fertilizing material.

To promote formation of the imidazolinone-counterion complex, the plant growth regulating compositions may be prepared with no or substantially no counterion (cation) chelating agent. Divalent cation chelating agents include EDTA (ethylenediaminetetraacetic acid), HEDTA (hydroxyethyl ethylenediamine triacetic acid), diethylenetrinitrilopentacetic acid (DTPA), 1,3-diamino-2-propanoltetraacetic acid (DTPA), and ethyleneglycolaminoethylestertetraacetic acid (EGTA). The plant growth regulating compositions may be prepared with no or substantially no ammonium sulfate, ammonium phosphate, or citric acid in order to promote formation of the imidazolinone-counterion complex.

As used in the present invention, surfactant can be used for several purposes. One possible advantage of a surfactant may be to increase efficacy of a plant growth regulator composition as described, including increasing one or more of: a plant growth regulator effect, residual (extended) plant growth regulator effect, herbicide effect, or residual (extended) herbicide effect. Other potential benefits of surfactant may include reduced phytotoxicity or structural damage to turf. In addition to these functional advantages, the use of surfactant may also be desired to allow or improve the ability to prepare a composition for storage, transport, or application to turf.

A very large variety of surfactants are known and commercially available, including such different classes as anionic surfactants, nonionic surfactants, cationic surfactants, ionic surfactants, and amphoteric surfactants, etc. According to the present description a surfactant can be any surfactant or combination of surfactants (e.g., blends of anionic, nonionic or cationic surfactants), useful to produce a plant growth regulator composition as described.

Generally, anionic surfactants include surface-active molecules in which an active portion of a lipophilic portion of the molecule forms a negative ion (i.e., anion) when placed in aqueous solution. Exemplary anionic surfactants include phosphoric acid ester surfactants (sometimes referred to as “phosphate ester” surfactants), sodium alkyl naphthalene sulfonate surfactants, and ethoxylated tristyrylphenol phosphate salts.

Exemplary sodium alkyl naphthalene sulfonate surfactants include sodium butyl naphthalene sulfonate, sodium di-n-butyl naphthalene sulfonate, sodium diisopropyl naphthalene sulfonate, sodium dimethyl naphthalene sulfonate, and mixtures thereof. Sodium butyl naphthalene sulfonate is commercially available, for example, under the trade name “MORWET B” from Witco/Crompton, Greenwich, Conn. Sodium di-n-butyl naphthalene sulfonate is commercially available, for example, under the trade name “MORWET DB” from Witco/Crompton, Greenwich, Conn. Sodium diisopropyl naphthalene sulfonate is commercially available, for example, under the trade name “MORWET IP” from Witco/Crompton, Greenwich, Conn. Sodium dimethyl naphthalene sulfonate surfactant is commercially available, for example, under the trade name “SELLOGEN HR” from Henkle Corp., Cincinnati, Ohio.

A nonionic surfactant is a surface-active molecule that does not contain ionizable polar end groups but does contain hydrophilic and lipophilic portions. Exemplary nonionic surfactants include polyoxyethylene alkylether or alkenylether surfactants. Nonionic surfactant used to prepare a suspension concentrate as described herein may include long or short chain alcohol ethoxylate surfactant. The alcohol ethoxylate surfactant may be branched or linear.

An example of a useful nonionic polyoxyalkylene surfactant includes alcohol ethoxylate having the general formula:

R—O—((CH₂)ₓO)_{y}—H

wherein R may be “long” or “short” chain and “branched” or “linear” alkyl. R preferably can be a “short chain” branched or linear alcohol, meaning that it can have from about 3 to 23 or fewer carbon atoms. With respect to the oxyalkylene, x can preferably be in the range from about 2 to 5, preferably from about 2 to 4 (e.g., 2 or 3, for a polyoxyethylene or polyoxypropylene, respectively) and y can preferably be in the in the range from 5 to 25.

Examples of useful short chain nonionic polyoxyalkylenes include linear alcohol polyoxyethylenes having the general formula:

CH₃(C₂H₄)ₘO(C₂H₄O)ₙH

wherein CH₃(C₂H₄)ₘ is a short chain linear alkyl having from about 3 to 23 or fewer carbon atoms (i.e., m can be in the range from about 1 to 11 carbon atoms), and n is in the range from about 5 to 25.

Another example is short chain nonionic polyoxypropylenes having the general formula:

CH₃(C₂H₄)ₘO(C₃H₆O)ₙH,

wherein CH₃(C₂H₄)ₘ is a short chain linear alkyl having from about 3 to 23 or fewer carbon atoms (i.e., m can be in the range from about 1 to 11 carbon atoms), and n can preferably be in the range from about 5 to 25.

Exemplary short chain linear alcohol ethoxylate surfactant are commercially available, for example, under the trade names “SURFONIC L12-6” from Huntsman, Austin, Tex., “SURFONIC L24-7” from Huntsman, Austin, Tex., “TERGITOL 15-S-7”, “TERGITOL 24-L-60”, “ALPHONIC 1012-60”, “ALPOHONIC 1414-60”, “BIOSOFT ET 630,” from Stepan Company, Chicago, Ill., and “GENOPAL 24-L-60.”

Other exemplary surfactants include polyethylene glycol, fatty acid ethoxylates, phosphate esters, octyl phenol ethoxylates, and nonyl phenol ethoxylates.

A cationic surfactant is a surface-active molecule in which an active portion of a lipophilic portion of the molecule forms a positive ion (i.e., cation) when placed in aqueous solution. In one embodiment, exemplary cationic surfactant includes ethoxylated tallow amine.

A plant growth regulator composition, or precursor, can include surfactant in any useful amount, including any amount that improves application or efficacy. Particular amounts can depend on factors related to the use of the plant growth regulator composition. For example, useful amounts of surfactant can depend on the intended application (including the turfgrass species applied to, or potential weed species affecting the turfgrass), the mode of application (e.g., field spraying or application from a hand-held spray applicator, or other technique), the method of any preparation of a plant growth regulator composition, the type of surfactant, the amounts and identities of other ingredients included in the plant growth regulator composition, etc.

Useful amounts of surfactant in a composition as described may be in the range from zero or close to zero to about 50 parts by weight surfactant based on the 100 parts by weight of the composition, e.g., up to 25 weight percent surfactant, preferably up to 15 or 10 parts by weight surfactant, or as low as 5, 2, 1, or 0.5 parts by weight surfactant based on 100 parts by weight of the composition.

Plant growth regulating compositions as described can be an application composition, or any of various types of precursor compositions. Exemplary application compositions can be aqueous, e.g., with water but a dry or any other non-aqueous liquid medium that allows equilibrium between the metal ion and the imidazolinone ion can be used. A precursor composition may be any of a variety of known types of wet, dry, solid, liquid, mixtures of these, dispersions, suspensions, etc, including compositions sometimes referred to as wettable powders, water dispersible granules, granules, aqueous solutions, water soluble powders, emulsifiable concentrates, oil-based flowables, concentrated emulsions, suspo-emulsions, emulsions, suspensions, suspension concentrates, mixtures, dispersions, microemulsions, impregnated pellet or other solids such as clay-based or paper-based materials, seed mats, soil preparations, biodegradable plastics, seeding materials, hydroseeding materials, or irrigation materials such as drip or overhead.

Plant growth regulator compositions as described, including precursor and application compositions, can be formed by any useful known methods, or by methods that will be developed. Generally, methods include combining a form of imidazolinone such as an imidazolinone acid or salt, with a cation (e.g., metal cation) in the form of a salt. These ingredients can be of any form and may be prepared and provided as any technical source, preferably a source suitable for use in an agricultural application.

In some embodiments of compositions that involve an aqueous solution, a salt of the imidazolinone ion is dissolved in the presence of a dissolved salt of the stabilizing cation (e.g., a metal salt if the cation is a metal ion). In the aqueous solution, the free imidazolinone ion and the free stabilizing counterion (e.g., metal ion) will become associated in the form of the complex, which, while in an aqueous environment, will remain in equilibrium with its counterpart ionic constituents according to Equation I above.

An imidazolinone ion can come from any source, such as an imidazolinone salt as described herein, and as are commercially available. A counterion such as a metal cation can also be available in any of a variety of salt forms that will dissociate upon being placed in an aqueous environment, for complexing with the imidazolinone ion. Examples of useful sources of divalent metal cation capable of dissociating in an aqueous environment include salts of divalent metals, for example salts of Cu, Fe, Mn, Mg, Ca, Ti, Cr, Co, Ga, and Al.

The process of combining the imidazolinone ion with the stabilizing counterion to form a complex can take place at any desired location. For example ingredients can be combined at a manufacturing site and the combined materials can be transported for sale and use in the form of a complex. Alternately, sources of the imidazolinone ion and counterion can be combined at any stage preceding application to turf, up to and including mixing these components in a tank used for application (i.e., “tank mixing”), optionally with addition of other ingredients of an application composition such as any one or more of an additional plant growth regulator, herbicide, insecticide, fungicide, fertilizer, surfactant, or a pH adjusting amount of an acid. For example, one embodiment of a plant growth regulator composition as described can be prepared by tank mixing to combine a composition containing the described complex with a preemergent herbicide (e.g., pendimethalin, prodiamine, etc.).

An application composition or a precursor can be at any useful pH. For example, the composition has a pH of greater than 2, such as in the range of about 2-10, or greater than 3.6, such as in the range of about 4-10.

A plant growth regulator composition, e.g., an application composition, can be applied to turfgrass in any of countless commercial and residential applications, to name a few: commercial and residential lawn cover; golf courses; athletic fields; parade grounds; parks; roadsides; managed turf like home lawns, around offices, around warehouses, sports complexes, sports fields (everything from football to baseball to soccer to cricket), municipalities, turf farms, school campuses, roadsides, hotels, gated communities, airports, military installations, community centers, green belts, medians, rest areas, state parks, national parks, berms, ditches, or any turf that is managed for its appearance or maintained in any way such as sprayed, mowed, watered, fertilized, or classified as commercial or residential turf, or for floor management in orchards, vineyards, nursery's, livestock yards, coffee plantations, banana plantations, power stations, Christmas trees. Post or soil applications to ornamentals or small trees or shrubbery, flowers, or hedges can be performed. The composition may be applied to rehabilitate lawn that has been allowed to become deteriorated, or a lawn that may be unimproved but in transition or possible transition to improved. Effects, as described, include growth regulation such as can be exhibited by seed head suppression, internode stacking, lateral and vertical growth reduction. The compositions may also be used in combination with either or both of post- and pre-emergent weed control, as salvage treatment, or in turf renovation, as a foliar fertilizer, inducing green up, improvement in structural aspects, increasing of verdure.

Turf that can be treated includes any types of turf. The terms “turf” and “turfgrass” interchangeably refer to grass plants considered to be any of a “turf,” a “grass,” or a “turfgrass,” as these terms are conventionally used to refer to relevant plant species, including cool season grasses and warm season grasses, the terms also excluding cultivated plants or crop plants. These and other turf species include a multitude of varieties adapted to specific conditions of precipitation, temperature, and sun and shade tolerance. The two basic categories are cool season grasses and warm season grasses. Also included are any turf species that may be considered “transitional” in nature and turf species that require a “winter preparation” treatment.

“Warm” season turf can be considered as all species, varieties, biotypes of Zoysia, Bermuda, Byhalia grass, centipede, St. Augustine, Kikuyugrass, Bentgrass, Paspalum, Buffalograss, or any other species which may be considered “warm season” turf by experts. Specific warm season species and hybrids include Tifway 419 Bermuda, TifEagle Bermudagrass, MiniVerde™ Ultra Dwarf Bermudagrass Celebration Bermuda Grass, SeaIsle 1 Paspalum, Platinum TE Paspalum SeaDwarf Seashore Paspalum, Meyer zoysia, Palmetto St. Augustine, and TifBlair Centipede. “Cool” season grasses can be considered species, varieties, biotypes of bluegrass, fescues, bentgrass, ryegrass or any other grasses considered “cool” season by experts in the field. Still other categories of turfgrass include Bahiagrass, Centipedegrass, Carpetgrass, and Grama grass, as well as others. As used herein, turfgrass also includes any modified, varied, or genetically altered species as Roundup™ tolerant species or HPPD tolerant species

Ornamental grasses are distinguished from turf grass and include those from the family Poaceae (sometimes referred to as “true grasses”) include perennial bunch (or tussock) grasses; sedges and bamboo are also considered ornamental grasses. Examples of ornamental grasses include Pampas Grass, Blue Oat grass, Deer Grass, Tufted Hair-grass, and Canyon Prince Wild Blue Rye.

“Plant health” can be described in relation to one or more of the following characterictics: greenup onset (i.e., when turf takes on a greener color due to the start of growing season), dormancy onset, return from dormancy, resistance to climate and diseases, stress tolerance, appearance and structure. Healthy turf typically exhibits normal or early greenup onset, normal or delayed dormancy onset, an easy return from dormancy, resistance to climate and diseases, and a good appearance and structure.

A plant growth regulating composition as described, including imidazolinone and metal ion, in a complex, can applied to turf in a manner by which the composition acts as a plant growth regulator, i.e., to inhibit growth of turf, preferably with reduced or limited observable phytotoxicity or other types of damage to the turf. Inhibiting turf growth refers to a reduced growth activity of a turfgrass plant to a degree that the plant exhibits substantially reduced growth. Inhibited growth can be measured by any objective basis, such as by measuring height, measuring horizontal growth, seed production, or by visual observation, by observation of inhibited internode elongation, observation of plant morphology, or observation of underground or above ground reproduction structure (without the plant dying); turf growth inhibition can be either short term or long term growth regulation and may be exhibited as seed head suppression, internode stacking (e.g., a reduction in vegetative matter between internodes), lateral growth suppression, vertical growth suppression, dormancy inducement, fruit (seed) abortion, metabolism reduction, amino acid inhibition, or suppression of chlorophyll production. A useful measure of growth inhibition can be different for different types of turf.

Horizontal growth includes “spreading out” of the turf above the surface of the soil. The plant growth regulating composition can inhibit horizontal growth above the surface, which reduces the need for edging and trimming. Inhibiting the horizontal grown can also prevent the turf from growing in unwanted areas

Lateral growth includes the spreading out of roots in the soil in a non-vertical direction. Inhibiting lateral grown can also reduces the need for edging and trimming, as well as preventing turf from growing in unwanted areas

In some aspects, growth suppression of turf using the plant growth regulating composition can be expressed relative to a growth of turf using a control composition. The assessment can be performed under the same growing conditions, with the exception of the addition of the complexed imidazolinone component, and any optional component, such as surfactant, etc. Suppression of growth can be determined at a particular time following application of the composition, such as at 1, 2, 3, 4, 5, 6, 7, or 8 weeks after application of the composition. In some aspects, suppression of growth (vertical, or horizontal, or both) of a treated area is about 25% or greater, about 50% or greater, as compared to a control. For example, a level of about 50% suppression in vertical growth of turf happens when at a selected time after application, the turf treated with the plant growth regulating composition has a height about half of turf not treated with the composition.

For example, for vertical growing grass such as bluegrass and similar types, a measure of growth inhibition can be vertical growth. Turf growth may be considered to be inhibited if turf exhibits vertical growth (as measured by any standard measure) of less than one centimeter per week. Alternately, for other types of turfgrass such as Bermuda grass, turf growth may be considered to be inhibited if turf fails to produce seeds (by visual inspection) for a period of time following application of the complex, such as for at least 1, 2, 3, 4, 5, 6, 7, or 8 weeks after application.

As described, and while not wishing to be bound by theory, imidazolinone ion is an effective plant growth regulating agent, yet the complex formed by an imidazolinone ion and a stabilizing counterion such as a metal cation is not effective as a plant growth regulating agent, yet is stable to a useful degree. Methods of the present description exploit these properties of imidazolinone ions and imidazolinone ion-stabilizing counterion complexes. According to exemplary methods, a plant growth regulator composition that contains the described imidazolinone ion-stabilizing counterion complex, can be applied to turf. A portion (e.g., a small fraction) of the complex is immediately dissociated to free ionic forms of the counterion and imidazolinone ion, allowing the dissociated imidazolinone ion to function as a plant growth regulator to inhibit the growth of the turf, optionally also to function as a herbicide to protect against growth of unwanted species or weeds. Another amount of the total amount of the complex applied to the turf environment, such as within the soil near plant roots, will remain in the form of the complex. This amount of the complex dissociates over time, such as over a period of weeks, to gradually release dissociated imidazolinone ion that continues to exhibit biological function including as plant growth regulator and optional herbicide, over the release period. By releasing imidazolinone ions from imidazolinone ion-stabilizing counterion complex gradually, over a period of time, the complex acts as a sort of time-release mechanism for the active imidazolinone ion plant growth regulator compound resulting in extended or residual turf growth inhibition and preferably herbicide activity.

Further, after application of the plant growth regulating composition, a portion of the amount applied can be in contact with leaf portion (e.g., grass blades) of the plant tissue, and an amount can be in contact with soil. The portion that is in contact with the leaf portion can be made immediately available to the plant and thereby that portion can immediately provide a growth regulating effect. The other portion is present in the soil and over time, the complex can dissociate and be taken up by the root system of the plant, thereby more slowly providing a growth regulating effect, as described herein. If desired, one or more additional step can be taken to increase the portion of imidazolinone complex in the soil. For example, the turf can be watered soon after application of the plant growth regulating composition to drive a greater amount of imidazolinone complex off the grass blade and into the soil. Another way of providing a greater amount of the imidazolinone complex in the soil is to make the composition dilute.

According to preferred plant growth regulating compositions (“application compositions”) and related methods, the complex is sufficiently stable in a dirt or soil environment so that an amount of the complex is present and gradually dissociates over a release period. The release period may be days or preferably weeks, resulting in continuous availability during the release period of an amount of the active imidazolinone ion (dissociated from the complex) to result in effective plant growth regulation (i.e., inhibition as described). The release period can correspond to a desired period of growth inhibition (“inhibition period”), which can be different depending on factors such as the type of turf, growing season, application rate, and which can for example be a period of up to several weeks, such as at least 2 or 4 or 6 weeks, preferably up to 8 or 10 weeks.

In preferred methods of use, the complex can be applied to avoid phytotoxicity and other damage to turf, such as root damage or other structural damage. At least in part the reduction in these negative impacts may be due to the application of imidazolinone in the form of a complex; the composition can include a sufficient amount of imidazolinone ion (as the complex) to function to inhibit turf growth for the entire inhibition period. If this amount were applied directly to turf in the ionic form, not in the complex with a cation, negative effects such as phytotoxicity or physical damage to turf plants may result. By use of the complex to release imidazolinone ion to the turf environment gradually, over time, the amount of active imidazolinone ion present during a release or inhibition period can preferably be maintained below a concentration that would cause such phytotoxicity or other plant damage.

A complex can be applied to turf at any useful application rate, which can be a rate useful to result in plant growth regulator effect as measured by growth inhibition and optional weed control, and preferably to result in a growth regulator effect while also avoiding phytotoxicity and other damage to turf. An example of an application rate may be in the range from 0.01 to 1.0 pounds imidazolinone (acid equivalent) per acre, e.g., from 0.05 to 0.8 pounds per acre, or from 0.1 to 0.4 pounds per acre.

The plant growth regulator composition can be applied by any method and in any manner, optionally with adjuvants such as surfactants, additional herbicide, insecticide, or fungicide compositions, or fertilizer, and optionally sequentially. Examples of methods for application may include spraying by any method, by irrigation, with fertilizer or other liquid or solid or semi-solid compositions, directly to turf or to soil that is prepared for seeding, sodding, or sprigging, as a small number of examples. The composition can be applied at any useful time, such as during a summer growing season to reduce the need for mowing, or following or ahead of a growing season such as in the fall or spring when turf is otherwise dormant.

If applied during a growing season, steps can include application followed by a period of inhibited growth and reduced need for mowing. If the growing season is sufficiently long, multiple applications (or “application cycles,” e.g., 2 or 3, depending on the inhibition period) can be applied, with a recovery period between applications to allow turf to grow normally for a period of 1, 2, or 3 weeks, with normal mowing and maintenance. For instance a first application cycle may include steps of mowing turf; waiting (during a “wait period”) for a desired condition of the turf, for example waiting for turf to grow back a marginal amount after mowing to “fill in”; applying plant growth regulating composition; suspending mowing for a period of weeks during which turf growth is inhibited (an inhibition period); when desired, such as after the inhibition period, mowing the turf regularly. If a second application can be used during a single growing season, the second application can preferably be applied after a recovery period, during which the turf is maintained and mown normally. Steps can be preformed during non-dormant conditions.

A wait period as described is optional. The need for a wait period can be based on factors such as the type of turf and the condition of the turf after mowing. A wait period can allow a freshly-mown turf to recover and achieve the condition and look that the user wants the turf to exhibit during the growth-inhibited stage (inhibition period)—so the turf is in a desired or optimal condition during the period of inhibited growth. For certain species, such as Bermuda grass, a wait period may be desired to let the turf grow for a couple of days after mowing, to fill in. After the grass has filled in, to look more full or thick, a growth regulator can be applied and growth can be inhibited.

As an example of a method as described, with multiple applications in a single growing season, can include the following steps: mow turf; optional wait period of from 0 to 6 days, depending on the type of turf; apply growth regulator composition; suspend mowing a period of at least 2 weeks, optionally from 4 to 6 weeks, up to 8 or 10 weeks, depending on the type of turf, growing conditions, etc.; after the inhibition period, mow lawn regularly for a period of weeks, e.g., 2-3 weeks, to allow the grass to grow and recover to a more normal, non-regulated condition. Re-apply; wait an optionally wait period as above, and suspend mowing for an inhibition period, as long as growth is inhibited. This application cycle, with two applications separated by a recovery period, may be sufficient to control growth of turf during a single growing period, e.g., over a single summer.

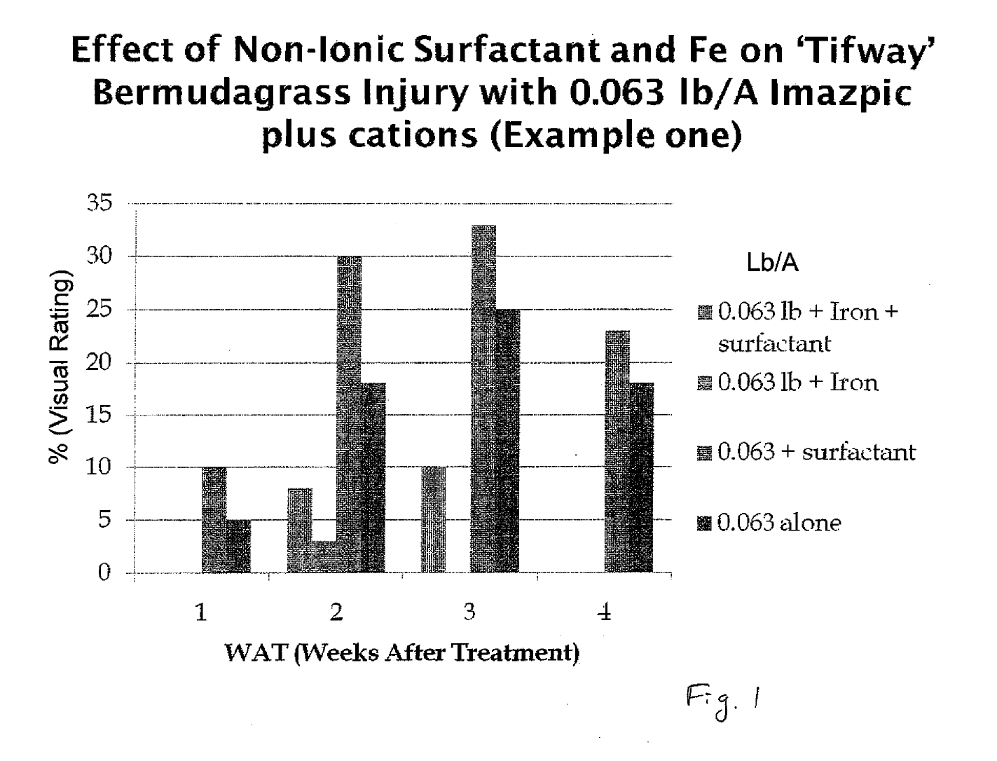

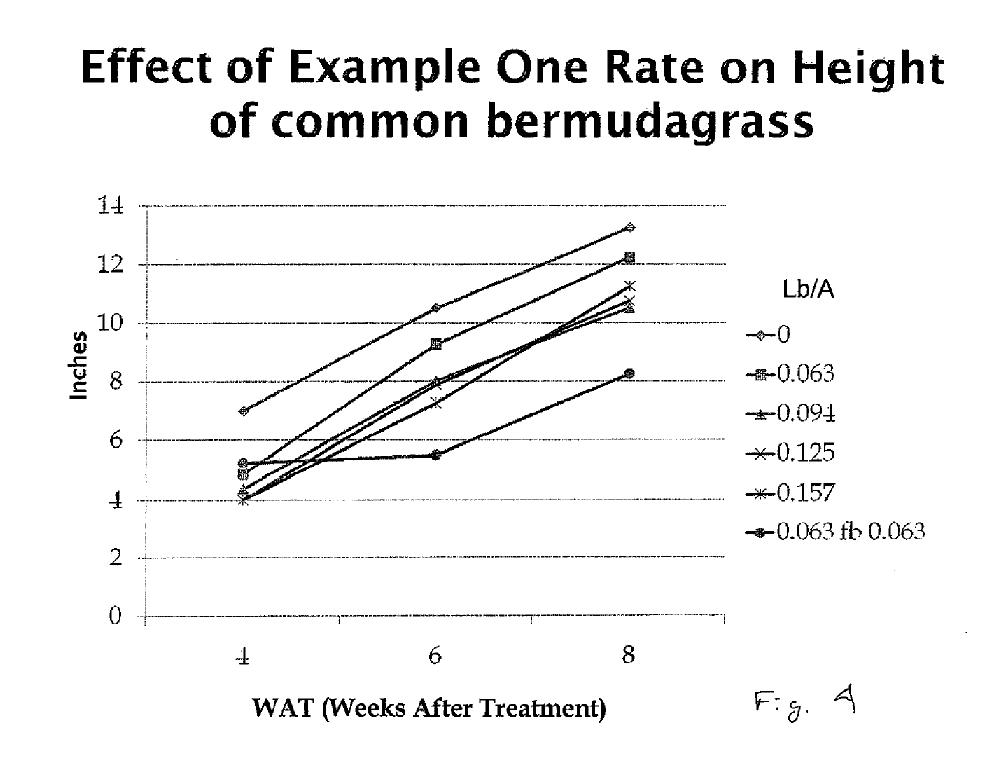

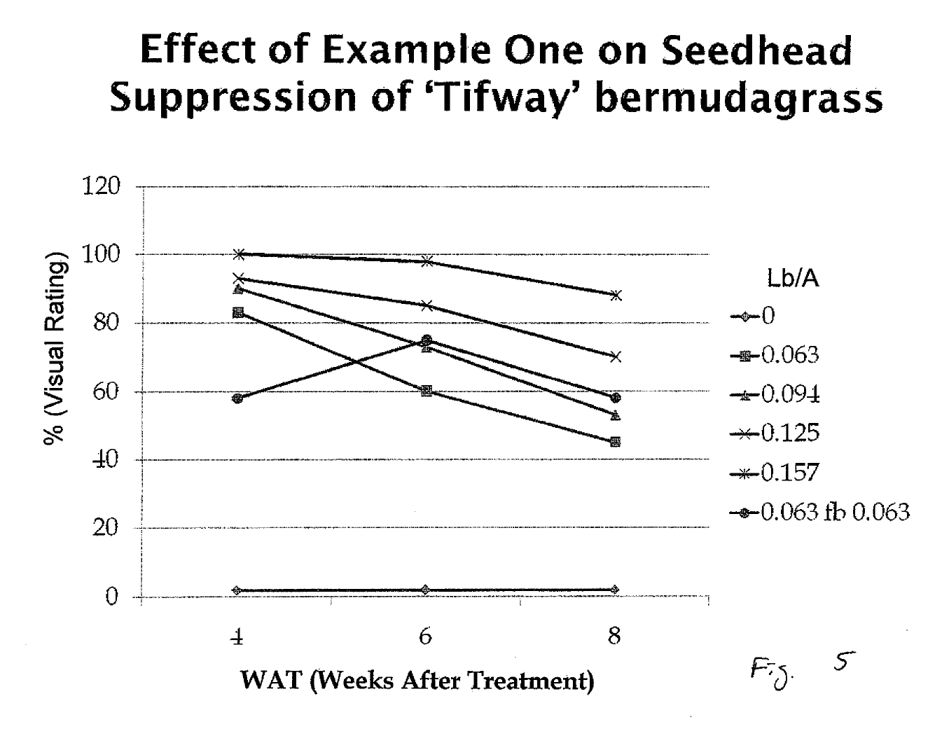

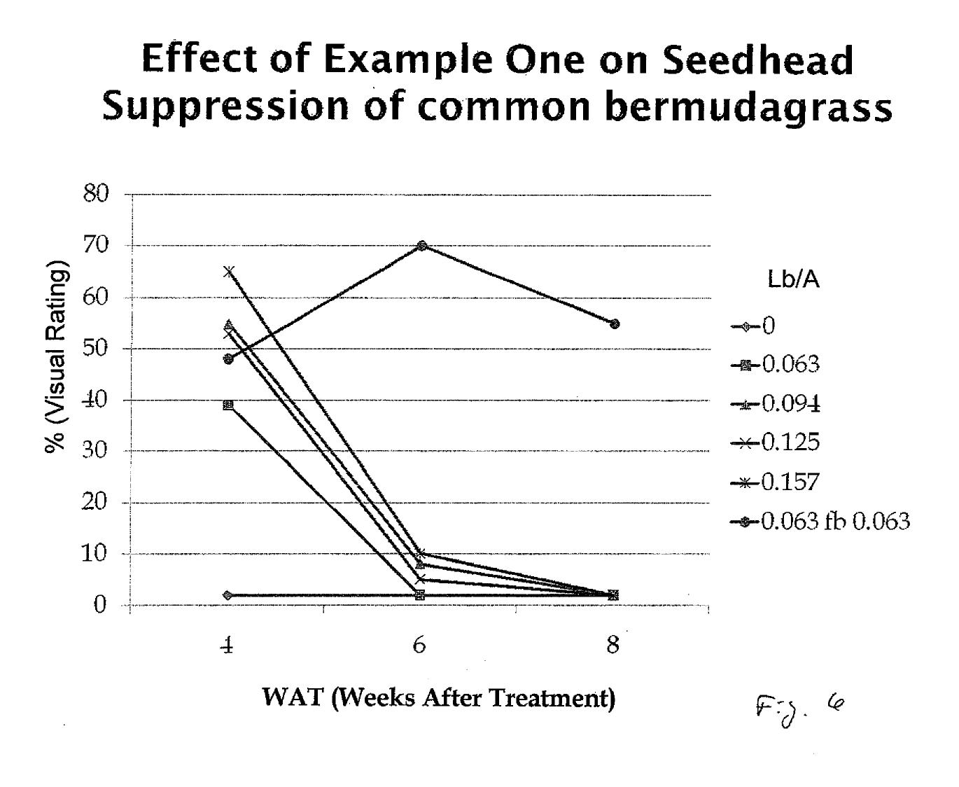

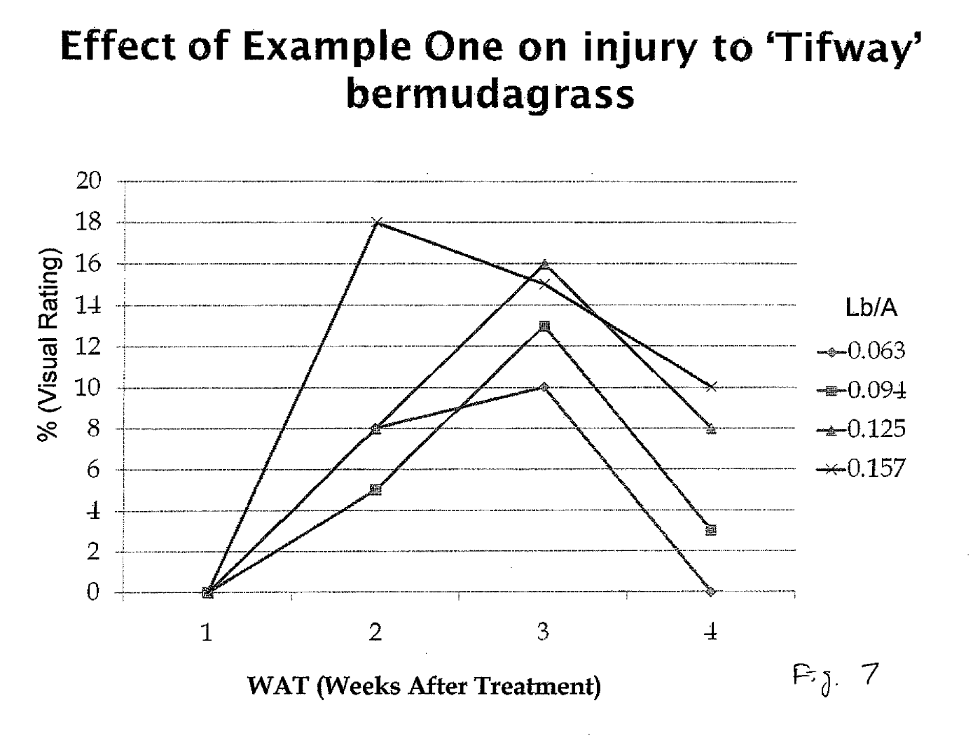

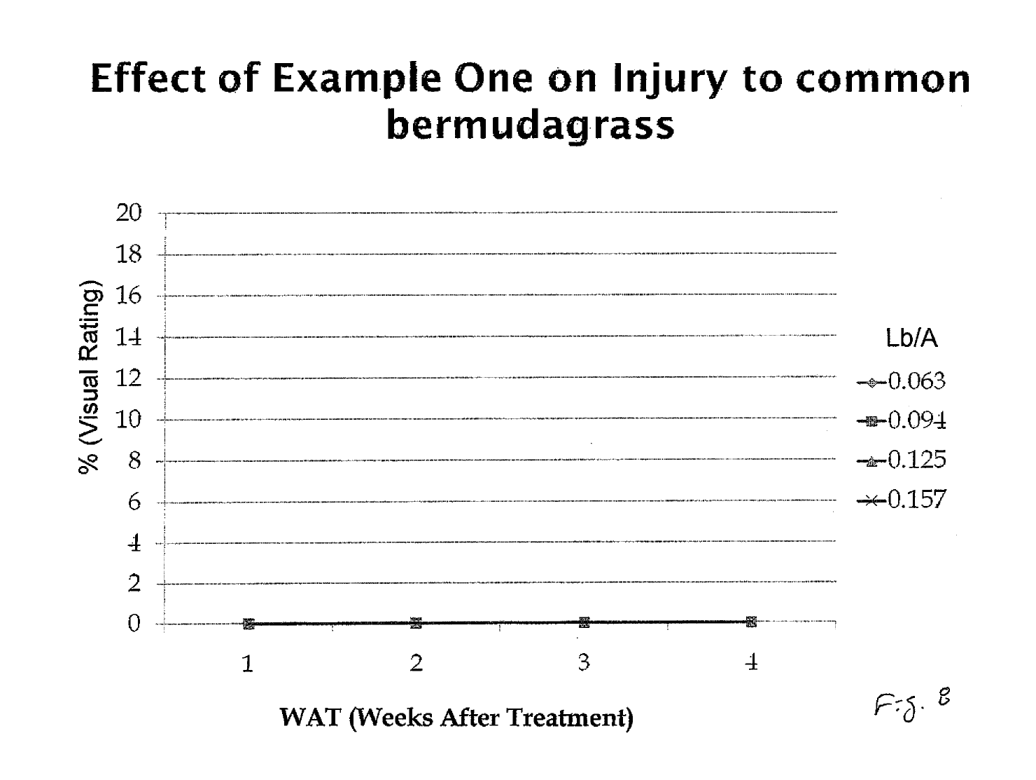

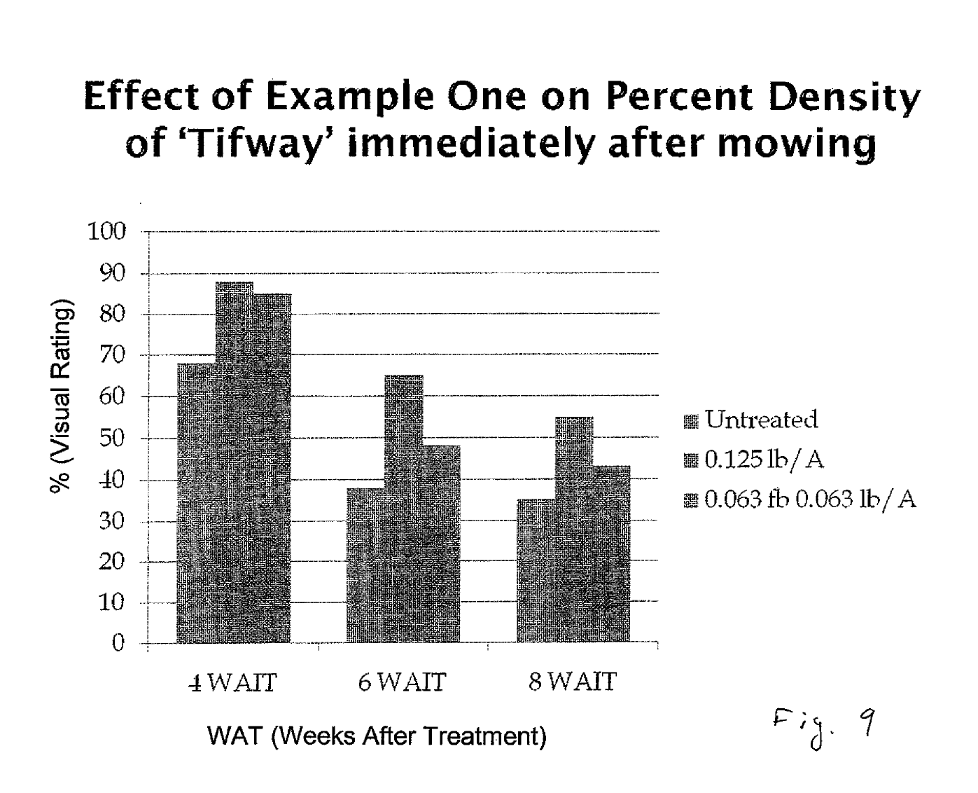

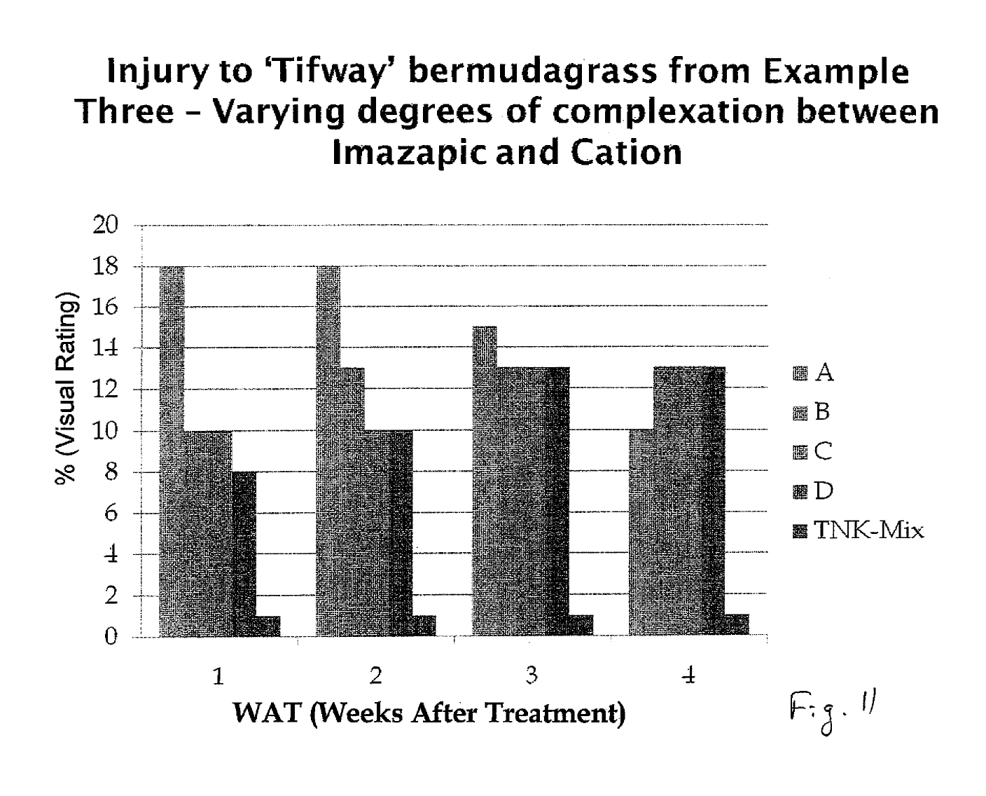

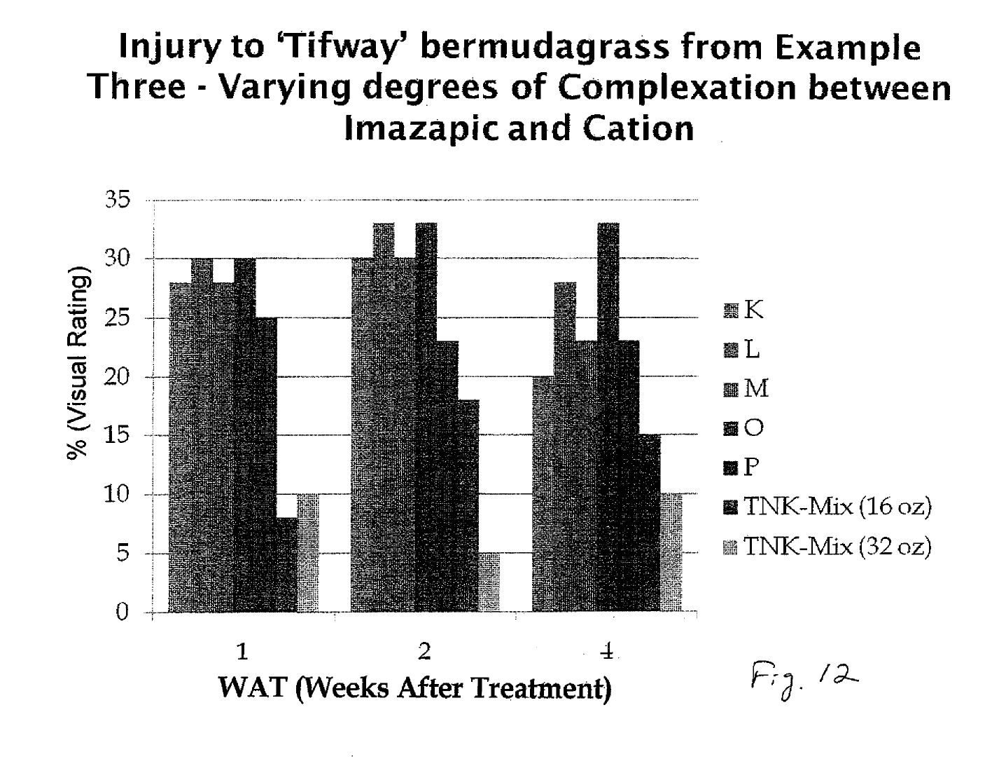

FIGS. 1 through 12 show information related to results of methods of the invention involving applying imidazolinone ion in an imidazolinone-metal ion complex, to turf.

FIG. 1 shows levels of reduced phytotoxicity upon application of a complex that includes imidazolinone ion and metal cation.

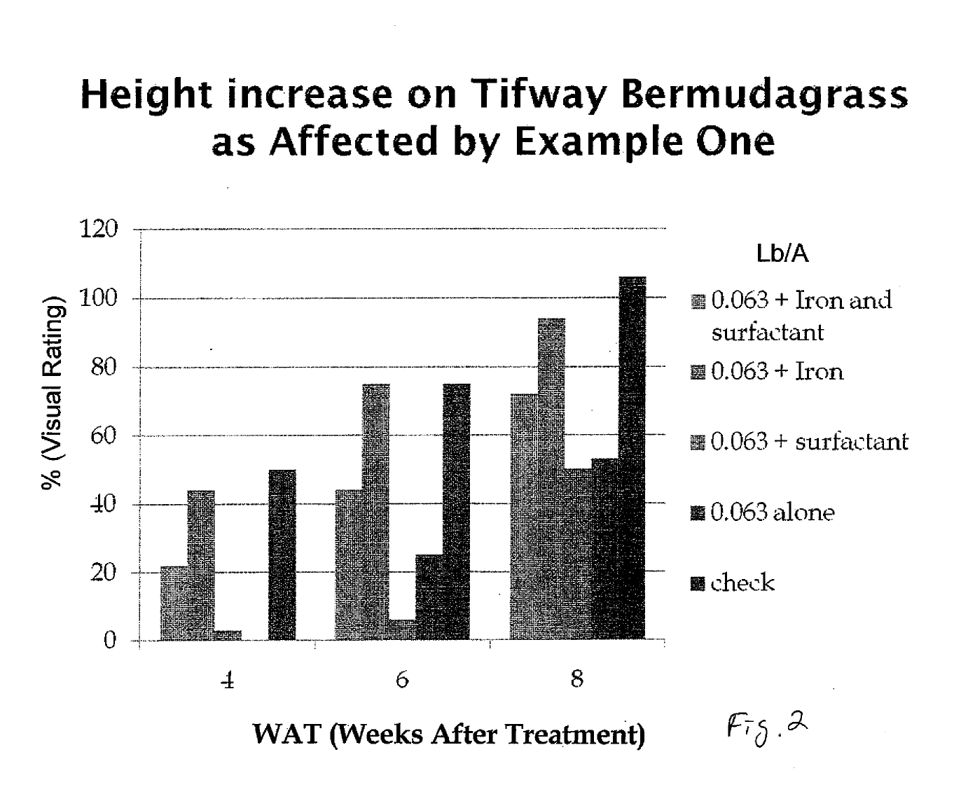

FIG. 2 shows growth inhibition upon application of a complex that includes imidazolinone ion and metal cation.

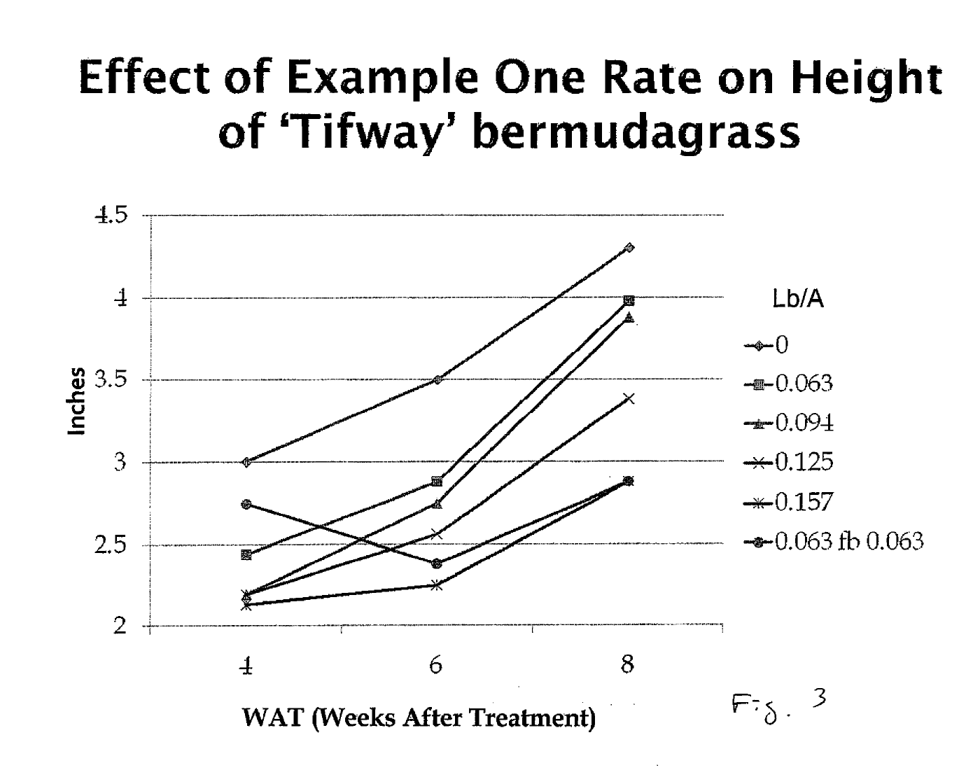

FIGS. 3 through 9 show growth measurements, seedhead suppression, injury, and percent density, upon use of an imidazolinone-metal ion complex with various types of turf.

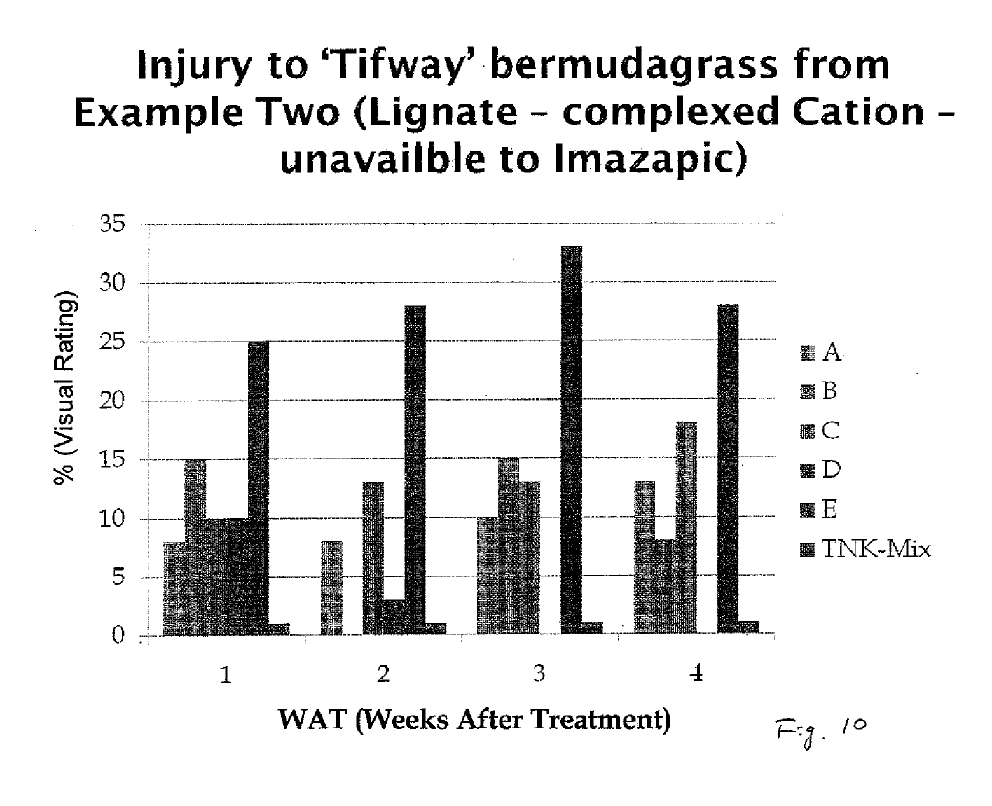

FIGS. 10 through 12 show results of applying imidazolinone-metal ion complex, with varying amounts of metal ion.

The foregoing description relates to the following exemplary, non-limiting compositions and methods:

What is claimed is:

1. A method of regulating growth of turf or ornamental grass by application of a plant growth regulator composition, the method comprising
providing a plant growth regulator composition comprising a complex of imidazolinone ion and counterion (e.g., stabilizing counterion),
applying the plant growth regulator composition to turf or ornamental grass in an amount effective to inhibit growth of the turf.

2. The method according to claim 1 comprising applying the plant growth regulator composition to turf in an amount effective to inhibit growth for at least 2 weeks.

3. The method according to claim 1 comprising applying the plant growth regulator composition to turf in an amount effective to inhibit growth of the turf for at least 4 weeks.

4. The method according to claim 1 comprising
providing a plant growth regulator composition comprising an imidazolinone-metal ion complex,
mowing a lawn comprising turf,
waiting a wait period of from 0 to 6 days following the mowing to allow the turf to grow,
after the wait period, applying the plant growth regulator composition to turf in an amount effective to inhibit turf growth,
suspending mowing of the lawn for a period in the range from 4 to 6 weeks.

5. The method according to claim 4 wherein the wait period is from 1 to 5 days and the turf is bluegrass.

6. The method according to claim 4 comprising
after the suspending step, mowing the turf during a recovery period,
after the recovery period, applying the plant growth regulator composition to turf in an amount effective to inhibit turf growth,
suspending mowing of the lawn a period in the range from 4 to 6 weeks.

7. The method according to claim 1 wherein the complex comprises
imidazolinone ion selected from imazethapyr ion, imazaquin ion, imazapyr ion, imazamethabenz ion, imazapic ion, imazamox ion, and imazaquin ion, and
metal counterion selected from a copper ion, an iron ion, a manganese ion, a magnesium ion, a titanium ion, a chromium ion, a cobalt ion, a gallium ion, and an aluminum ion.

8. The method according to claim 1 wherein the counterion is divalent copper or iron.

9. The method according to claim 1 wherein the composition comprises a surfactant.

10. The method according to claim 1 wherein the composition comprises
imidazolinone ion in an amount in the range of 1 to 10 parts by weight, and
metal counterion in an amount in the range of 0.5 to 5 parts by weight.

11. The method according to claim 1 wherein the imidazolinone ion and metal counterion are in amounts in the composition in a molar ratio in the range of 2:1 to 1:64.

12. The method according claim 1 wherein the imidazolinone ion and counterion are in amounts in the composition in a molar ratio in the range from 1:1 to 1:32.

13. The method according to claim 1 comprising applying imidazolinone ion (acid equivalent) in an application rate in the range of 0.01 to 1 pound per acre.

14. The method according to claim 1 wherein the turf is a warm weather turf.

15. The method according to claim 1 wherein the turf is selected from: Zoysia, Bermuda, St. Augustine, Kikuyugrass, Bentgrass, Paspalum, Buffalograss, Bluegrass, Ryegrasses, Fescues, Bahiagrass, Centipedegrass, Carpetgrass, and Grama grass.

16. The method according to claim 1 wherein the turf is bluegrass and the growth is inhibited to prevent more than 1 centimeter of growth for a period of at least a week.

17. The method according to claim 1 wherein the turf is Bermuda grass and the growth is inhibited to prevent seed formation for a period of at least 1 week.

18. A turf or ornamental grass growth regulating composition comprising imidazolinone ion and counterion as a complex.

19. The growth regulating composition according to claim 18 wherein the composition can be applied to turf or ornamental grass and soil, whereupon the complex releases imidazolinone ion in an amount to inhibit growth over a release period.

20. The composition according to claim 18 comprising
imidazolinone ion selected from imazethapyr ion, imazaquin ion, imazapyr ion, imazamethabenz ion, imazapic ion, imazamox ion, and imazaquin ion, and
metal counterion selected from a copper ion, an iron ion, a manganese ion, a magnesium ion, a titanium ion, a chromium ion, a cobalt ion, a gallium ion, and an aluminum ion.

21. The composition according to claim 18 wherein the counterion is divalent copper.

22. The composition according to claim 18 comprising
from 1 to 10 parts by weight imidazolinone ion, and
from 0.5 to 5 parts by weight metal counterion.

23. The composition according to claim 18 comprising amounts of imidazolinone ion and counterion in a molar ratio in the range from 2:1 to 1:64.

24. The composition according to claim 18 comprising surfactant.

25. The composition according to claim 24 comprising from 0.1 to 50 parts by weight surfactant based on 100 parts by weight of the composition.

26. The composition of claim 1 wherein the imidazolinone ion is predominantly an S chiral isomer, or is predominantly an R chiral isomer.

27. A method of forming a growth regulating composition comprising steps of:
providing an imidazolinone salt or acid,
providing a salt comprising a metal ion, and
combining the imidazolinone salt and the salt comprising the metal ion.
